(19) United States
(12) Patent Application Publication
(10) Pub. No.: US 2014/0275903 A1
(43) Pub. Date: Sep. 18, 2014

(54) SYSTEM AND METHOD FOR QUICK-ACCESS PHYSIOLOGICAL MEASUREMENT HISTORY

(71) Applicant: LIFESCAN SCOTLAND LIMITED, Inverness-shire (GB)

(72) Inventors: David ELDER, Inverness-shire (GB); Antony Smith, Dingwall (GB)

(73) Assignee: LifeScan Scotland Limited, Inverness-shire (GB)

(21) Appl. No.: 13/826,663

(22) Filed: Mar. 14, 2013

(51) Int. Cl. A61B 5/00; A61B 5/021; A61B 5/145

(52) U.S. Cl. CPC A61B 5/742; A61B 5/14532; A61B 5/021; A61B 5/14542
USPC 600/365; 600/300; 600/485; 600/309

(57) ABSTRACT

Described are methods and systems to allow the use of a very simple physiological meter without a user input interface (i.e., buttonless) while maintaining the ability to store time linked measurement records for retrospective or prospective analysis of the measured physiological measurements with a bistable display to allow for a display of plural prior physiological measurements without necessitating the activation (e.g., turning on or manipulation of the user input interfaces) of the physiological meter.

### BACKGROUND

Physiological measurements can be performed with a wide variety of known physiological measurement devices. For example, body temperature, cardiac rhythm, blood pressure, oxygen saturation in blood, electrocardiography, EEG, pulse, skin conductance, total hemoglobin, carboxyhemoglobin, methemoglobin, perfusion index and the like can be monitored with small handheld instrument or meter. Similarly, physiological measurements can be made of analytes (glucose, ketone, cholesterol and the like) present in physiological fluids, e.g. blood or blood derived products. Physiological detection find use in a variety of applications, including clinical laboratory testing, home testing, hospitals, clinical, etc., where the results of such testing play a prominent role in diagnosis and management in a variety of disease conditions.

One area that applicants have concentrated is the physiological monitoring of persons with diabetes. In such person, glucose monitoring is one technique to ensure normal glycemic state of such person. The accuracy of such monitoring can significantly affect the health and ultimately the quality of life of the person with diabetes. Generally, a diabetic patient measures blood glucose levels several times a day to monitor and control blood sugar levels. Failure to test blood glucose levels accurately and on a regular basis can result in serious diabetes-related complications, including cardiovascular disease, kidney disease, nerve damage and blindness. There are a number of electronic devices currently available which enable an individual to test the glucose level in a small sample of blood. One such glucose meter is the OneTouch® Profile™ glucose meter, a product which is manufactured by LifeScan.

There currently exist a number of portable electronic devices that can measure physiological parameter(s) (e.g., body temperature, cardiac rhythm, blood pressure, oxygen saturation in blood, electrocardiography, EEG, pulse, skin conductance, total hemoglobin, carboxyhemoglobin, methemoglobin, perfusion index, glucose levels, ketone, cholesterol and the like) in an individual and store the measurements for recalling or uploading to another computer or remote processor for analysis. These devices are provided with user input interfaces such as buttons and capacitive touchscreen to allow the user to manipulate information or configure parameters for the meter.

It has been proposed by others in the art to utilize a buttonless physiological meter, as shown and described in U.S. Pat. No. 5,410,474, which is incorporated by reference herein. From the standpoint of the users, a meter without any button or user input interface is very attractive due to its operational simplicity. Others have proposed a disposable meter that utilizes a bistable display in US Patent Application No. 2012/0053436. Nevertheless, such systems are susceptible to various modes of inefficiency or error.

### SUMMARY OF THE DISCLOSURE

Applicants have recognized that a person managing a chronic disease (e.g., diabetes, asthma, high blood pressure and the like) with a buttonless physiological monitor described earlier faces the problem of accessing that person's prior physiological measurements quickly and intuitively in the absence of any user input interface (buttons, touchscreen, or voice-command and the like). Another problem identified by applicants is that, without a user input interface (e.g., buttons, touch screen, voice or visual command interfaces), a user would not be able to access his or her prior historical measurement results. Furthermore, an physiological meter without any user input interface would not allow for setting of temporal parameter(s) such as time, date or both time and date. For the manufacturer of such meter, there is a substantially lower cost of manufacturing because the deletion of a user input interface (e.g., buttons, touch screen or non-contact touchscreen). However, a meter without a user input interface does not allow for manipulation of the temporal parameters (e.g., time, date or both time and date). Consequently, any stored physiological measurement will not have a time record or time-stamp to indicate when the physiological measurement was taken. This would render the physiological measurement records virtually worthless without the measurements being linked to the appropriate temporal parameters. And even if a user input interface is provided, the ability to obtain prior physiological results is often complex and counterintuitive, thereby rendering such system less useful to a person managing a chronic disease.

In one aspect, a physiological measurement device is configured without any user input interface is provided. The device includes a microprocessor, memory, bistable display coupled to each other. The bistable display of the device is controlled by the microprocessor to present plural prior physiological measurements stored in the memory when no power is provided to the bistable display. The plural prior physiological measurements may include a graphical representation of such physiological measurements previously conducted over a predetermined time period.

In a further aspect, a method of operating a physiological measurement device has been devised by applicants. The device has a microprocessor coupled to a memory and a display. The method can be achieved by: conducting an physiological measurement of a user; displaying the physiological measurement value after the conducting step; storing the physiological measurement value into the memory; shutting down the device; detecting one of: (a) shaking of the device subsequent to the terminating step, or (b) activation of an user input interface; and displaying at least one prior physiological measurement upon the detecting step being established.

In another aspect, a method of operating a physiological monitoring system having a physiological meter with a microprocessor linked to a clock, memory, bistable display, and configured such that the meter is without any user input interface for a user to set temporal parameters for the physiological meter. The method can be achieved by: determining whether the clock has been reset, and if true, evaluating whether a clock reset flag has been set; if the evaluating step is false then setting a clock reset flag and setting the clock to its initial factory parameters, otherwise if the evaluating step is true then disqualifying any physiological measurement record having a delta-time flag associated with the record; if the determining step is false then querying as to whether a physiological measurement has been made; if the querying is true, ascertaining as to whether a clock reset flag has been set; if the querying is false then storing the physiological measurement linked to a record of a current temporal parameter of the clock otherwise if the querying is true then storing the physiological measurement with both a delta-time flag and a current temporal parameter of the clock; verifying whether a clock reset flag is set and if the clock reset flag is not set, displaying the time at which the physiological measurement was taken on the bistable display of the meter with power to the processor turned off otherwise if the clock reset flag is set, prohibiting a display of the time at which the measurement was recorded on the bistable display of the meter when power to the processor is turned off.

Other variations in any of the aspects described above are possible. For example, the plural physiological measurement may include a last five measurement results; the physiological measurement may include blood glucose; the physiological measurement may include blood pressure; the physiological measurement may include blood oxygen saturation; the biosensor may include a glucose test strip. The device may have another display disposed in an overlaid manner over the bi-stable display. The another display is configured to display a current physiological measurement and the bi-stable display is configured to display at least one physiological measurement prior to the current physiological measurement.

As another example, one of the methods may further include establishing whether the meter is in communication with another or remote processor that has its own clock and if the meter is in communication with the another processor, checking if the clock reset flag is set; and if the checking returns a true then calculating a differential time between the local time clock of the remote processor and the clock of the meter and linking all stored physiological measurement records with a differential time flag using the differential time from the calculating step, otherwise if the checking returns a false then checking to see if a temporal adjustment between the clock of the meter and the remote processor is needed. Alternatively, the determining step may include checking for at least one internal error of the clock circuit or any circuitry of the meter; the determining step may include checking for electrostatic discharge in the clock circuit, interruption in clock oscillation, or any fault or interruption in the circuitry of the meter; the displaying may include a display of at least one recent physiological measurement; the prohibiting may include a display of at least one recent physiological measurement. In the method, the temporal parameters may include date and year; the physiological measurement device may include a blood glucose meter and the detecting may include inserting a test strip into a test strip port of the meter.

In the aforementioned aspects of the disclosure, the steps disclosed may be performed by an electronic circuit or a processor. These steps may also be implemented as executable instructions stored on a computer readable medium; the instructions, when executed by a computer may perform the steps of any one of the aforementioned methods.

In additional aspects of the disclosure, there are computer readable media, each medium comprising executable instructions, which, when executed by a computer, perform the steps of any one of the aforementioned methods.

In additional aspects of the disclosure, there are devices, such as test meters or analyte testing devices, each device or meter comprising an electronic circuit or processor configured to perform the steps of any one of the aforementioned methods.

These and other embodiments, features and advantages will become apparent to those skilled in the art when taken with reference to the following more detailed description of various exemplary embodiments of the invention in conjunction with the accompanying drawings that are first briefly described.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention (wherein like numerals represent like elements).

FIG. 1A illustrates a preferred blood glucose measurement system with a physiological meter for use with an analyte biosensor in the form of a disposable test strip.

FIG. 1B illustrates a variation on the system of FIG. 1A in which a conventional display is utilized with a bistable display.

FIG. 2 illustrates the various components disposed in the meter of FIG. 1A.

FIG. 3 illustrates the logic to allow for time record linkage to the physiological measurement record for a meter configured without a user input interface.

### MODES FOR CARRYING OUT THE INVENTION

Applicants' invention has achieved the goal of allowing persons to use a very simple meter (i.e., one without any user input interface such as buttons, touch screen or voice recognition interface) with virtually none of its disadvantages when it comes to keeping track of physiological measurements for prospective or retrospective analysis of the analyte measurements. Therefore, the following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. In addition, as used herein, the terms “patient,” “host,” “user,” and “subject” refer to any human or animal subject and are not intended to limit the systems or methods to human use, although use of the subject invention in a human patient represents a preferred embodiment.

FIG. 1A illustrates a diabetes management system that includes a meter 10 and a biosensor in the form of a glucose test strip 18. Note that the meter 10 may be referred to as a physiological measurement and management unit, a glucose meter, a meter, and a physiological measurement device. In an embodiment, the meter unit may be combined with an insulin delivery device, an additional analyte testing device, and a drug delivery device. The meter unit may be connected to a remote computer or remote server 21 via a cable (not shown) or a suitable wireless technology 20 such as, for example, GSM, CDMA, BlueTooth, WiFi and the like.

Referring back to FIG. 1A, meter unit 10 may include a housing 12 a display 14, and a strip port opening 16 to receive a biosensor. The display 12 can be configured to show the temporal parameters 14a, physiological measurement 14b and past recorded physiological measurements that can be presented statistically in graphical form. The electronic components of meter 10 may be disposed on a circuit board 34 that is within housing 12.

Of note in the meter 10 is display 14 which is in the form of a bistable display. As is known by those skilled in the art, the bistable display is a liquid crystal display in which the crystals may exist in one of two stable orientations. The result is that the bistable display retains the image produced without being powered or with extremely low power. The display 14 is connected to a display driver (not shown) which is coupled to the microprocessor. The display driver and associated hardware for driving and controlling the bistable display is widely available commercially, such as for example from Lumex Inc., Focus Display Solutions Inc., Dalian Good Display Co., Ltd., ZBD Displays Ltd., Kent Displays Inc., to name a few. The bistable display 14 is controlled by the microprocessor via the display driver to present at least one prior physiological measurement stored in the memory when no power is provided to the bistable display. Generally, the prior physiological measurement may include a last result of a physiological measurement provided in display area 15a. Alternatively, instead of the last measurement in display area 15a, a plurality of measurements can be displayed. In the example shown here, the last five physiological results are in the form of blood glucose concentration, indicated as 127 mg/dL; 97 mg/dL; 113 mg/dL; 85 mg/dL; and 78 mg/dL. Although the meter 10 is shown as a blood glucose meter, other types of physiological meters can also utilize this technique devised by applicants. For example, the meter can be in the form a blood pressure monitor and the physiological measurements can be blood pressure taken during different times of the day; the meter can be in the form of a pulse oximetry meter with oxygen saturation values being the physiological values measured at various times of the day. Alternatively, the meter may include more than one physiological monitor. It should be clear that the technique devised by applicants here are not limited to the few examples described herein. And depending on the type of physiological measurements being obtained, there can be from 3 to 30 of the prior results which can be shown on the bistable display.

The bi-stable display 14 can also include display area 15b in which the average measurement over a predetermined time period can be displayed. In the example of FIG. 1A, display area 15b shows an average physiological measurement over the last 24 hours of 100 mg/dL. Where the meter has been configured to provide more details, the bistable display 14 can be configured to have display portion 15c which plots the results in portion 15a graphically. Although a two-dimensional graph is shown here in portion 15c, other form of graphical representations can be utilized such as, for example, a pie-chart.

In another embodiment, shown here in FIG. 1B, instead of a single bistable display, the display of meter 10 can be configured as two different displays mounted in an overlaid fashion to one another. One of the displays (e.g., 13) can be a conventional display whose images are not retained when power is removed from the display, display driver or device. The other display (e.g., 14) can be a bistable display. During normal operation, the top display 13 can be configured so that images on the lower display are not visible when the top display 13 is powered by the driver. When power is removed from display 13, the images on bistable display 14 would be visible. Prior to a physiological measurement being made, a last measurement can be displayed on bistable display 14. Once the current measurement is made, it can be displayed on display 13 and the value of the current measurement is substituted for the last measured value on bistable display 14 so that the next time a measurement is made, the current measurement (now the “last” measurement prior to the “next” measurement) would be visible to the user without the user needing to scroll through or activating multiple sequences of the user input interface (buttons or voice command) to get at the prior or last measurements.

In a further alternative where no bistable display is utilized, applicants have devised the following technique to allow a user to quickly access prior measurement records. In this technique, when the device detects one of: (a) shaking of the device subsequent to the terminating step, (b) analyte test strip insertion into a test strip port of the device, or (c) activation of a user input interface such as a single button, the device automatically display at least one prior physiological measurement upon the detecting of one of (a) and (b).

FIG. 2 illustrates (in simplified schematic form) the electronic components disposed on a top surface of circuit board 34. On the top surface, the electronic components include a strip port connector 22, an operational amplifier circuit 35, a microcontroller 38, a display connector 14a, a non-volatile memory 40, a clock circuit 42, and a first wireless module 46. On the bottom surface, the electronic components may include a battery connector (not shown) and a data port 13. Microcontroller 38 may be electrically connected to strip port connector 22, operational amplifier circuit 35, first wireless module 46, display 14 (which can be a bistable display or a combination of conventional display and bistable display in a stacked or overlaid fashion), non-volatile memory 40, clock 42, battery, and data port 13.

Operational amplifier circuit 35 may include two or more operational amplifiers configured to provide a portion of the potentiostat function and the current measurement function. The potentiostat function may refer to the application of a test voltage between at least two electrodes of a test strip 18. The current function may refer to the measurement of a test current resulting from the applied test voltage. The current measurement may be performed with a current-to-voltage converter. Microcontroller 38 may be in the form of a mixed signal microprocessor (MSP) such as, for example, the Texas Instrument MSP 430. The TI-MSP 430 may be configured to also perform a portion of the potentiostat function and the current measurement function. In addition, the MSP 430 may also include volatile and non-volatile memory. In another embodiment, many of the electronic components may be integrated with the microcontroller in the form of an application specific integrated circuit (ASIC).

Strip port connector 22 may be configured to form an electrical connection to the test strip. Display connector 14a may be configured to attach to display 14. Display 14 may be in the form of a liquid crystal display for reporting measured glucose levels, and for facilitating entry of lifestyle related information. Display 14 may optionally include a backlight. Data port 13 may accept a suitable connector attached to a connecting lead, thereby allowing glucose meter 10 to be linked to an external device such as a personal computer. Data port 13 may be any port that allows for transmission of data such as, for example, a serial, USB, or a parallel port. Clock 42 may be configured to keep current temporal parameters related to the geographic region in which the user is located and also for measuring time. The meter unit may be configured to be electrically connected to a power supply such as, for example, a battery.

Strip 18 includes a reagent layer (typically glucose dehydrogenase (GDH) based on the PQQ co-factor and ferricyanide). In another embodiment, the reagent or enzyme may be replaced with the enzyme GDH based on the FAD co-factor. When blood or control solution is dosed into a sample reaction chamber of strip 18, glucose is oxidized by GDH₍ₒₓ₎ and in the process converts GDH₍ₒₓ₎ to GDH_{(red)}, as shown in the chemical transformation T.1 below. Note that GDH₍ₒₓ₎ refers to the oxidized state of GDH, and GDH_{(red)} refers to the reduced state of GDH.

D-Glucose+GDH₍ₒₓ₎ Gluconic acid+GDH_{(red)} T.1

Next, GDH_{(red)} is regenerated back to its active oxidized state by ferricyanide (i.e. oxidized mediator or Fe (CN)₆³⁻) as shown in chemical transformation T.2 below. In the process of regenerating GDH₍ₒₓ₎, ferrocyanide (i.e. reduced mediator or Fe(CN)₆⁴⁻) is generated from the reaction as shown in T.2:

GDH_{(red)}+2Fe(CN)₆³⁻ GDH₍ₒₓ₎+2Fe(CN)₆⁴⁻ T.2

Meter 10 may include electronic circuitry that can be used to apply a plurality of voltages to the test strip 18 and to measure a current transient output resulting from an electrochemical reaction in a test chamber of the test strip 18. The signal processor 38 of meter 10 is provided with a set of instructions for the method of determining an analyte concentration in a fluid sample.

As is known, the user inserts the test strip into a strip port connector of the test meter to connect at least two electrodes of the test strip to a strip measurement circuit. This turns on the meter 10 and the meter may recognize that the strip 18 has been inserted, the test meter 10 initiates a fluid detection mode. Once it has been determined that sufficient fluid amount has been deposited, the meter automatically initiate the glucose test. Details of this technique to determine sufficient volume for electrochemical testing are shown and described in U.S. Pat. Nos. 7,195,704; 6,872,298; 6,856,125 and 6,797,150, which documents are incorporated by reference as if fully set forth herein. A determination of the glucose concentration from the current transient output from the test strip 18 can be found in U.S. Pat. No. 7,749,371, patented Jul. 6, 2010, which was filed on 30 Sep., 2005 and entitled “Method and Apparatus for Rapid Electrochemical Analysis,” which is hereby incorporated by reference in its entirety into this application.

Referring to FIG. 3, applicant has devised a logical process 300 to allow for any physiological meter without a user input interface to utilize temporal parameters linked to physiological measurements and storing both the temporal parameters with the respective physiological measurements. Process 300 can be initiated whenever the meter is turned on, after a test measurement, or when connected to a remote processor, such as, for example, a personal computer, a smartphone, or a remote server 21. At step 302, a check is made by the microcontroller 38 whether at least one of a command to reset clock 42 or an occurrence of the clock 42 being reset has been made due to an error, a command or the application of power (e.g., such as during the insertion of a new battery). If step 302 returns a true, then microcontroller 38 evaluates as to whether a clock reset flag has been set at step 304. In the event the evaluating step 304 is false (or returning a “no”), then the controller 38 sets a “clock reset” flag as part of its program at step 310 and the clock 42 to its initial factory parameters at step 308. Otherwise if the evaluating step 304 is true then the system disqualify (at step 306) any physiological measurement record having a delta-time flag associated with the physiological measurement record. Thereafter, the clock 42 is set to its initial parameters. The initial parameters may include the temporal parameters provided to the system during manufacturing of the meter. This may include the initial date and time programmed into any non-erasable memory of the clock circuit. As used herein, the phrase “disqualify” and variations on this root term means that the disqualified physiological measurement records cannot be used or shown to the user even though such records are available for purpose of diagnostics.

If the determining step 302 is false then the system queries as to whether a physiological measurement has been made at step 312. If the querying is true, then the system ascertains, at step 314, as to whether a clock reset flag has been set. At step 314, if the querying is false then the system stores the physiological measurement linked to a record of a current temporal parameter of the clock at step 318, otherwise if the querying at step 314 is true then the system stores the physiological measurement with both a differential or “delta time” flag and a current temporal parameter of the clock at step 316; verifying whether a clock reset flag is set and if the clock reset flag is not set, displaying clock time on the display of the meter otherwise if the clock reset flag is set, prohibiting a display of the clock time on the display of the meter when the physiological measurement record is reviewed. Applicants note that where meter utilizes an audible annunciator (with or without the display), the annunciator is also prohibited from annunciating the temporal parameters for the physiological measurement records. As used herein, the phrase “current temporal parameter” of the clock is intended to include at least a current clock time for the geographic area in which the clock is located and preferably, current time, date and year for such geographic location.

If the query at step 312 returns a false then the system establishes at step 320 whether the meter is communication or preparing to communicate with a remote processor 21. If step 320 returns a true then the system checks to see if a “clock reset” flag has been set at step 322. If step 322 is true, a calculation is made at step 324 of ΔT where ΔT is a representation of a difference between the temporal parameter of the remote processor 21 versus the temporal parameter of the clock 42 of the meter. The symbol delta signifies that time stamp linked to the measurement record from one measurement to another correct relatively but not absolutely. Where the temporal parameter is in the form of hours or minutes (or even seconds), ΔT is a time differential between the remote processor 21 and the clock 42. Alternatively, where the temporal parameter is in the form of days, ΔT is a date differential between the remote processor 21 and the clock 42. Thereafter, at step 326, all stored physiological measurement records with the ΔT flag is adjusted with the calculated ΔT. For example, if the clock 42 is 2 hours faster than the remote processor clock then all records with the ΔT flag is subtracted by two hours; if the clock 42 is 4 hours slower than the remote processor clock then 4 hours are added to all records with the ΔT flag.

On the other hand, if the check in step 322 returns a false, meaning that the clock reset flag is not set then a check is made at step 328 to determine if a temporal adjustment is needed for the clock 42 based on the temporal parameters of the remote processor clock. For example, at step 328, if it is determined that the clock 42 is too fast, too slow or in a different time zone then a flag can be set or the clock 42 can be adjusted to have the same temporal parameters (e.g., time and date) as remote processor clock. This step 328 is intended to account for time drift or different time zones.

If step 320 cannot establish that the meter is in communication with the remote processor 21, verification is made at step 330 to determine if the clock reset flag was set. If step 330 returns a true, meaning that the clock reset flag was set, the system prohibits the meter from showing the temporal parameters (e.g., time or date) at step 332. If step 330 returns a false, meaning that there is no clock reset flag established, then the meter is allowed to display the temporal parameters at step 334. Both steps 332 and 334 revert to the main routine at step 336.

In the logic devised by applicant, the determining step 302 may include checking for at least one internal error of the clock circuit or an error in any circuitry of the meter or the processor. Such error may include electrostatic discharge in the clock circuit or any circuitry of the meter or the meter circuit. It is noted that the displaying of the temporal parameters may include a display of at least one recent physiological measurement. Alternatively, the prohibition of the temporal parameters may include prohibiting a display of at least one recent physiological measurement or in other words, prohibiting the display of temporal parameters when the physiological measurement value is displayed or annunciated. In the method, the temporal parameters comprise date and year. Additionally, the method described herein can be programmed into any suitable processor so that the steps of the method can be carried out by such processor.

Applicant notes that this heretofore new technique is also applicable to any physiological measurement of physiological parameters and is not limited to analyte (e.g., glucose) measurement of blood. Moreover, the technique has advanced the state-of-the art by allowing for the technical effects of a very simple meter (i.e., one without any user input interface) with virtually none of its disadvantages when it comes to keeping track of physiological measurements for prospective or retrospective analysis of the measurements.

Accordingly, while the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

What is claimed is:

1. A physiological measurement device configured without any user input interface, the device comprising:
a microprocessor coupled to a biosensor to measure at least one physiological measurement;
a memory coupled to the microprocessor;
a bistable display coupled to the microprocessor and controlled by the microprocessor to present plural prior physiological measurements stored in the memory when no power is provided to the bistable display, the plural prior physiological measurements including a graphical representation of such physiological measurements previously conducted over a predetermined time period.

2. The device of claim 1, in which the plural prior physiological measurements comprise a last five measurement results.

3. The device of claim 1, in which the physiological measurement comprises blood glucose.

4. The device of claim 1, in which the physiological measurement comprises blood pressure.

5. The device of claim 1, in which the physiological measurement comprises blood oxygen saturation.

6. The device of claim 4, in which the biosensor comprises a glucose test strip.

7. The device of claim 1, further comprising another display disposed in an overlaid manner over the bi-stable display, the another display configured to display a current physiological measurement and the bi-stable display configured to display at least one physiological measurement prior to the current physiological measurement.

8. A method of operating a physiological measurement device having a microprocessor coupled to a memory and a display, the method comprising:
conducting a physiological measurement of a user;
displaying the physiological measurement value after the conducting step;
storing the physiological measurement value into the memory;
shutting down the device;
detecting one of: (a) shaking of the device subsequent to the terminating step, or (b) activation of an user input interface; and
displaying at least one prior physiological measurement upon the detecting step being established.

9. The method of claim 8, in which the physiological measurement device comprises a blood glucose meter and the detecting step comprises inserting a test strip into a test strip port of the meter.
